(19) United States
(12) Patent Application Publication
(10) Pub. No.: US 2022/0016355 A1
(43) Pub. Date: Jan. 20, 2022

(54) A drive mechanism for an applicator for delivering multiple doses of medicament and a method of assembling said drive mechanism

(71) Applicant: NEMERA SZCZECIN SPÓLKA Z OGRANICZONA ODPOWIEDZIALNOSCIA, Szczecin (PL)

(72) Inventors: Alberto LOZANO PLATONOFF, Szczecin (PL); Mateusz WILCZEK, Szczecin (PL)

(21) Appl. No.: 17/297,769

(22) PCT Filed: Nov. 27, 2019
(86) PCT No.: PCT/EP2019/082683

§ 371 Date: May 27, 2021

(30) Foreign Application Priority Data
Nov. 28, 2018 (PL) P.427951

(51) Int. Cl. A61M 5/315

(52) U.S. Cl. CPC A61M 5/31585; A61M 5/31551; A61M 5/31511; A61M 2207/00; A61M 5/31501; A61M 5/31526; A61M 5/31581

(57) ABSTRACT

A drive mechanism for an applicator for delivering multiple doses of medicament, comprising an axially moveable piston rod (2) having a non-circular cross-section and an external thread, a mounting member (4) with one member of a pair of blocking means, said pair comprising a toothed rim (4a, 3b) and at least one ratchet arm (3a, 4b); a drive sleeve (3) having an internal thread coupled with the thread of the piston rod (2), the drive sleeve (3) provided on the distal end with the other member of said pair of blocking means. A method of assembling the drive mechanism of the invention, in which the drive sleeve (3) together with the piston rod (2) and the piston rod guide (5) into the mounting member (4) are inserted from the side of the distal end of the applicator.

The present application relates to a drive mechanism for an applicator for delivering multiple doses of medicament and a method of assembling said drive mechanism.

There are medicament applicators known in the art in which a dose of medicament is delivered by pushing it out from a container (cartridge) by means of a piston positioned within the cartridge, the piston being displaced axially by an elongated piston rod. The mechanisms of such applicators usually comprise rotatable elements rotating about a longitudinal axis and it is therefore necessary to convert this rotation into an axial displacement of the piston rod.

In the applicator described in the international application WO99/38554A1 a piston rod is rotated about the axis of the applicator by a drive sleeve engaged with the piston rod by cooperating grooves and projections. The piston rod has an external thread coupling it with a nut fixed in a housing, wherein the nut has an internal thread engaging the thread of the piston rod. Due to this engagement, the rotation of the piston rod driven by the drive sleeve results in the axial movement of the piston rod and a respective axial movement of a piston engaging the piston rod foot. As a consequence, a determined medicament dose is pushed out of a cartridge. The nut with the thread causing the piston rod to move axially is provided with a pawl wheel teeth on its internal surface, the pawl wheel teeth cooperating with pawls of the drive sleeve, which allows the drive sleeve to rotate only in one direction. Use of a ratchet mechanism is necessary for providing correct operation of a dose setting and injecting mechanism. Owing to this, the drive sleeve does not rotate during dose setting and dose correcting and in consequence there is no undesired leakage of the medicament from the cartridge as only the rotation of the drive sleeve may cause the axial movement of the piston rod and the medicament delivery.

A European application EP0937471 A2 discloses a mechanism for driving a piston rod which does not rotate during injection. The applicator described in this document has a piston rod with indentations and a piston rod guide constituted by integral projections formed on the internal side of its housing. The piston rod has an external thread engaging an internal thread of a nut which rotates during injection about a main axis of the device.

Therefore methods for driving a piston rod by means of two elements, one of which being fixed in a housing and the other being a sleeve arranged around the piston rod and rotatable about the longitudinal axis of the device, are known in the state of the art. In such arrangements, the piston rod has a thread and some formations such as an indentation or a flattening, cooperating with said two elements. There are two possible arrangements of such mechanism. In one arrangement the drive sleeve has a thread, whereas the fixed element is a piston rod guide which prevents the piston rod from rotating by means of its shape which is designed for cooperating with said indentations, flattenings or other shapes of the piston rod cross-section. Rotation of the drive sleeve results in axial displacement of the piston rod which does not rotate during injection. In an alternative configuration the drive sleeve is rotationally coupled with the piston rod (via indentation, flattening or other formations) and the rotation of the drive sleeve causes also the piston rod rotation. In such arrangement the fixed element has an aperture with an internal thread and the piston rod rotates in this aperture. Such engagement results in that a rotation of the piston rod induces its axial displacement and medicament delivery.

Ratchet mechanisms allowing the drive sleeve to rotate only in one direction are also used in the applicators. Owing to this, the drive sleeve does not rotate during dose setting and dose correcting and any undesired axial displacement of the piston rod other than injection is prevented. The cooperating ratchet elements are disposed on the drive sleeve and the fixed element guiding the piston rod.

Alternatively, one of these elements, i.e. the piston rod guide or a toothed rim, may form an integral part of a housing. However, such design makes the manufacture to the housing by an injection moulding difficult because of the complicated shape of the housing. This is particularly significant if the housing is to have additional elements disposed on its side opposite to the medicament delivery direction, for example a thread, projections or indentations or a dose indication window. It is similarly disadvantageous if the housing comprises an element preventing axial movement of the drive sleeve. The internal diameter of the drive sleeve is usually significantly smaller than the internal diameter of the housing, so such axial movement preventing element would need to have relatively large dimensions. However, the structures formed on a cylindrical body of the housing should be as small are possible.

Furthermore, in the state of the art solutions, the mechanism for guiding the piston rod is fixed in the housing once the housing has been engaged with a cartridge holder. Such engagement may be permanent (in the case of disposable applicators) or releasable in reusable devices in which case it may be used for cartridge exchange. However, it requires that the cartridge holder is mounted simultaneously with the guide mechanism and/or the guide mechanism works properly (i.e. converts rotation of a drive element into axial displacement of the piston rod) only when the cartridge holder is engaged with the housing.

The aim of the invention was to provide a drive mechanism for an applicator the assembly of which would be simple and would not require an element preventing axial movement of a drive sleeve to be formed on an internal surface of a housing of the applicator.

Another aim of the invention was to provide a drive mechanism for an applicator in which the initial axial position of a piston rod could be adjusted during assembly.

Still another aim of the invention was to provide a piston rod guiding mechanism which does not require engagement of the cartridge holder and the housing to work properly.

Another aim of the invention was to provide a drive mechanism for an applicator preventing undesired axial displacement of the drive sleeve in a proximal direction.

Still another aim of the invention was to provide a method of assembling a drive mechanism for an applicator which would be simple and would not require an element preventing axial movement of a drive sleeve to be formed on an internal surface of a housing of the applicator.

According to the invention, a drive mechanism for an applicator for delivering multiple doses of medicament is provided, the drive mechanism having a distal end facing the needle end of the applicator, the drive mechanism comprising:

an axially moveable piston rod having a non-circular cross-section and provided with an external thread,

a mounting member provided with one member of a pair of blocking means, said pair comprising a toothed rim and at least one ratchet arm;

a drive sleeve having an internal thread coupled with the external thread of the piston rod, the drive sleeve being provided on the side of the distal end with the other member of said pair of blocking means,

wherein the blocking means of the mounting member and the blocking means of the drive sleeve cooperate radially during rotation of the drive sleeve, the rotation causing translation of the piston rod.

The drive mechanism according to the invention is characterized in that the drive mechanism further comprises a piston rod guide provided with a central aperture in which the piston rod is axially movable and blocked in rotation, the piston rod guide and the mounting member being provided with coupling elements for preventing rotation of the piston rod guide relative to the mounting member.

Preferably, the mounting member is provided with the member of said pair of blocking means constituted by the internal toothed rim and the drive sleeve is provided with the member of said pair of blocking means constituted by the at least one ratchet arm.

Alternatively, the mounting member may be provided with the member of said pair of blocking means constituted by the at least one ratchet arm and the drive sleeve may be provided with the member of said pair of blocking means constituted by the external toothed rim.

The coupling elements are preferably constituted by a flange of the piston rod guide, the flange being provided at its circumference with at least one projection, and by at least one indentation of the mounting member.

The mounting member preferably comprises an internal resilient projection allowing the piston rod guide to be inserted into the mounting member from the side of the distal end of the applicator and preventing the piston rod guide from sliding back out of the mounting member.

The internal projection is preferably a circumferential shoulder.

According to the invention, a method of assembling the drive mechanism of the invention is provided, the method comprising the following steps:

a step of sliding the piston rod guide onto the piston rod;

a step of screwing the drive sleeve onto the piston rod;

a step of inserting the drive sleeve together with the piston rod and the piston rod guide into the mounting member.

The method according to the invention is characterized in that the step of the inserting the drive sleeve together with the piston rod and the piston rod guide into the mounting member is realized from the side of the distal end, and in that the drive sleeve is coupled with the mounting member by the pair of blocking means comprising a toothed rim and at least one ratchet arm while the coupling elements of the piston rod guide are simultaneously engaged with the mounting member, wherein

an axial position of the piston rod in relation to the drive sleeve is pre-set by rotation of the piston rod guide relative to the mounting member prior to the coupling of the blocking means and engaging the coupling elements.

The step of inserting the drive sleeve with the piston rod and the piston rod guide into the mounting member preferably involves overcoming resistance of the internal resilient projection of the mounting member.

By using the two separate elements i.e. the piston rod guide and the mounting member which are engaged as late as during assembly, it is possible to insert the drive sleeve into the drive mechanism from its distal side. Furthermore, there is no structure on the internal surface of the housing of the applicator, which would cooperate with the piston rod guide, the piston rod or the drive sleeve, therefore the manufacture of the housing by injection moulding is easier. Reduced technological requirements result in lower manufacturing cost and shortened injection moulding cycle.

Due to the fact that the piston rod guide is a separate element, it is possible to alter its angular position relative to the mounting member during assembly. It allows to modify the initial axial position of the piston rod, because its rotation, along with the rotation of the piston rod guide, causes also its axial displacement due to a threaded engagement with the drive sleeve.

Furthermore, the drive mechanism according to the invention may be inserted into the housing without use of any additional elements for blocking or stabilizing the mechanism in the housing. Particularly, there is no need to couple the housing with the cartridge holder in order to prevent the undesired axial or rotational movement of the piston rod guide or the drive sleeve.

The drive mechanism according to the invention, while featuring the above-mentioned advantageous features, allows to convert the rotation of the drive element into the axial displacement of the piston rod. Such mechanism may be used in an applicator regardless of its purpose and a source of an energy driving the piston rod. Particularly, the mechanism may be used in both disposable (i.e. discarded when the whole volume of medicament has been delivered from a cartridge) and reusable applicators in which a cartridge can be replaced. It may also be used in both automatic applicators, i.e. the force needed for rotating a drive element is exerted by a driving spring with previously stored energy, and manual applicators in which this force is exerted directly by a user, for example by pressing a button. Furthermore, it may be used in both variable dose and fixed dose devices.

Embodiments of the drive mechanism according to the invention are presented in the drawings, in which:

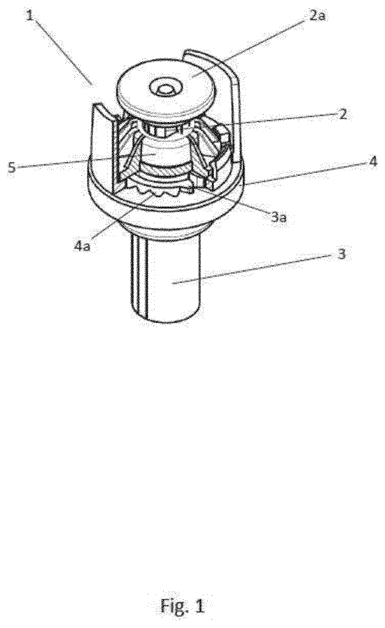

FIG. 1 shows a partially cross-sectional perspective view of the drive mechanism according to a first embodiment of the invention;

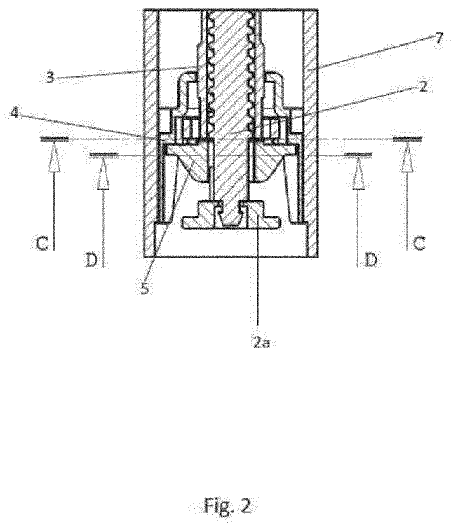

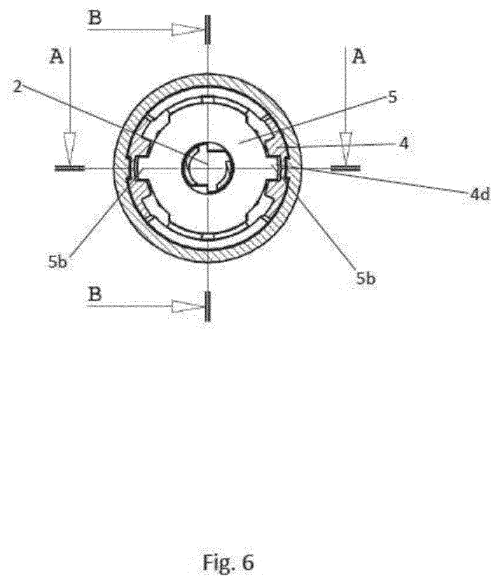

FIG. 2 shows a longitudinal section along a plane A-A indicated in FIG. 6 of a fragment of an applicator in which the drive mechanism is mounted;

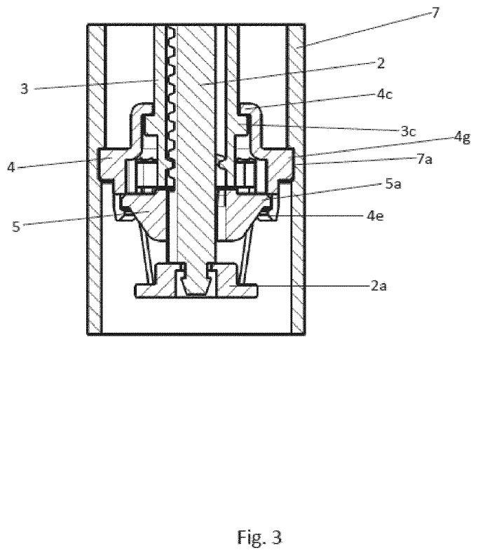

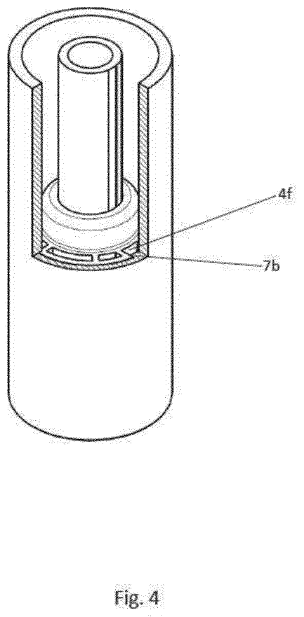

FIG. 3 shows a longitudinal section along a plane B-B indicated in FIG. 6 of a fragment of an applicator in which the drive mechanism according to the invention is mounted;

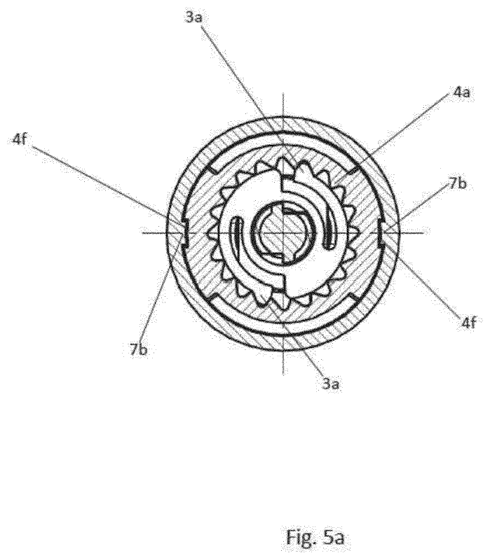

FIG. 4 shows a detailed view of a connection of the drive mechanism of FIG. 1 with an applicator housing;

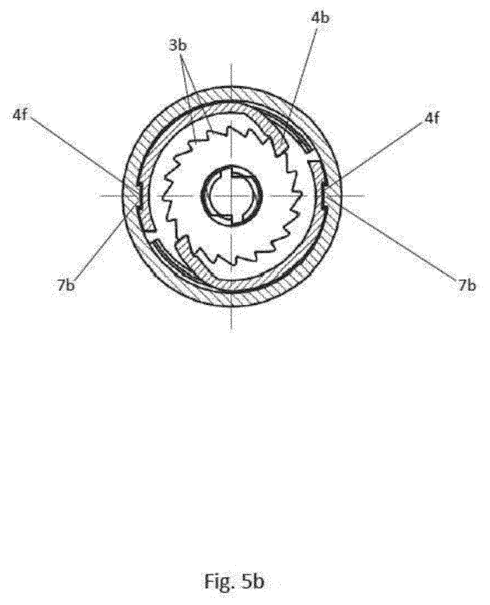

FIG. 5a shows a cross-section along a plane C-C indicated in FIG. 2 of the drive mechanism according to the invention placed in the housing:

FIG. 5b shows a cross-section along the plane C-C indicated in FIG. 2 of another embodiment of the drive mechanism according to the invention placed in the housing;

FIG. 6 shows a cross-section along a plane D-D indicated in FIG. 2 of the drive mechanism according to the invention placed in the housing;

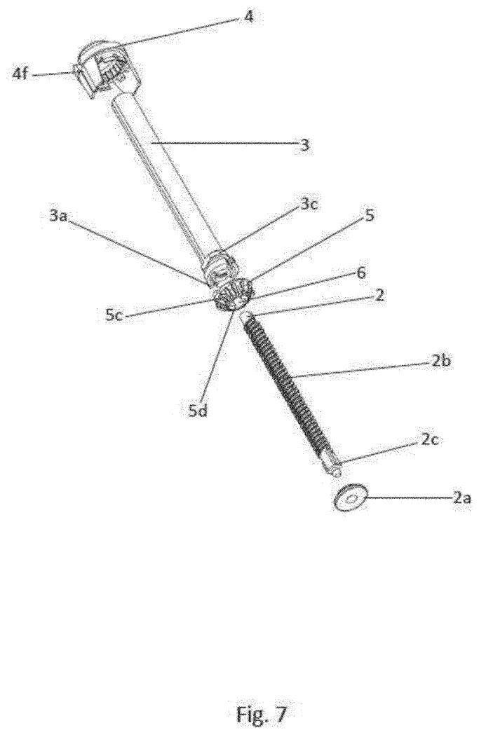

FIG. 7 shows an isometric view of the components of the drive mechanism according to the invention;

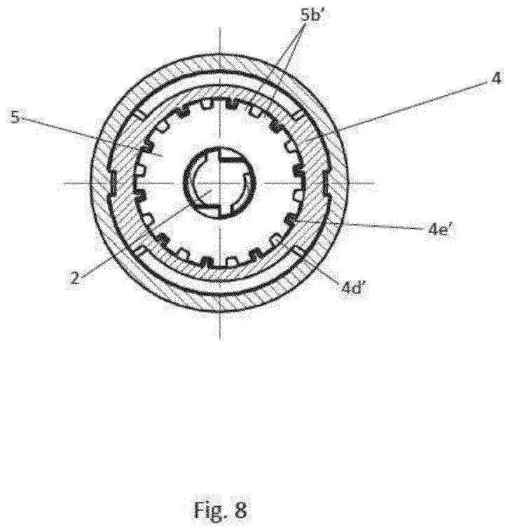

FIG. 8 shows a cross-section along the plane D-D indicated in FIG. 2 of still another embodiment of the drive mechanism according to the invention placed in the housing;

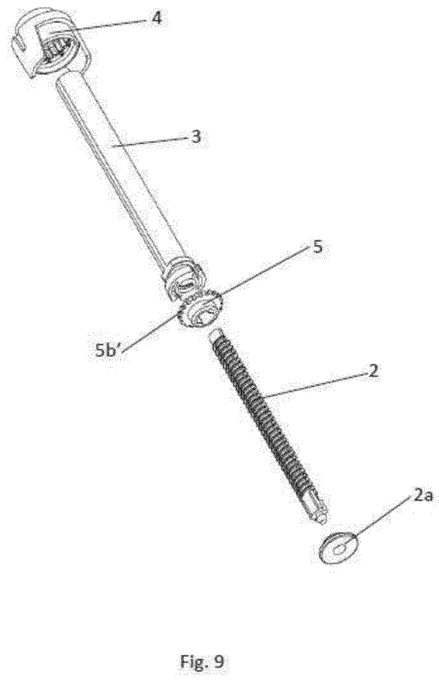

FIG. 9 shows an isometric view of the components of the embodiment shown in FIG. 8;

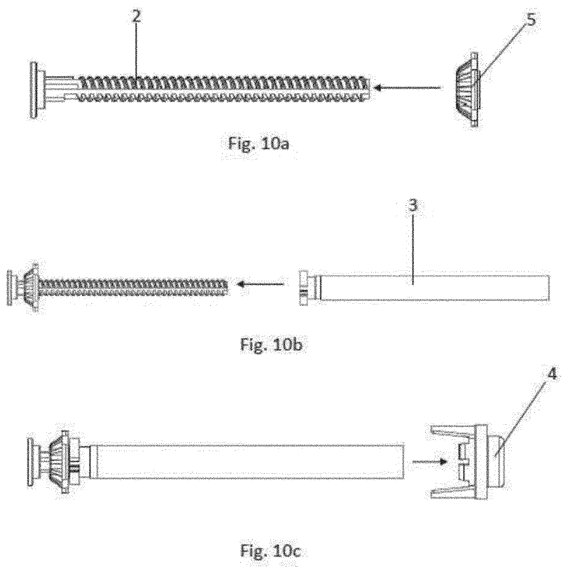

FIG. 10 shows a sequence of steps of the method of assembling the drive mechanism according to the invention.

In the following description the term “distal” relates to the end of the drive mechanism facing the needle side of the applicator in which it is mounted and the term “proximal” relates to the opposite end thereof.

The drive mechanism 1 is presented in FIG. 3 as assembled. In FIG. 1 the drive mechanism is shown as seen from its distal end and it comprises the following components: a piston rod 2 with a piston foot 2a at its end, a drive sleeve 3, a mounting member 4 and a piston rod guide 5. The same components are shown in the longitudinal sections of FIGS. 2-3, which present a fragment of an applicator with the drive mechanism mounted in a housing 7.

The piston rod 2 has a non-circular cross-section and it is provided with an external thread. After the assembly of the drive mechanism in an applicator, the piston rod 2 may be displaced only along a longitudinal axis of the applicator in a distal direction.

The drive sleeve 3 is screwed onto the piston rod 2 and it has an internal thread coupled with the external thread of the piston rod 2. Furthermore, the drive sleeve 3 has two resilient ratchet arms 3a at its distal end (FIG. 1 shows one of them).

As can be seen in FIG. 5a, the mounting member 4 has an internal toothed rim 4a. The resilient ratchet arms 3a and the toothed rim 4a form a pair of blocking means of the drive sleeve 3 and the mounting member 4. Cooperation of this pair of blocking means, i.e. the ratchet arms 3a of the drive sleeve 3 and the toothed rim 4a of the mounting member 4, allows to rotate the drive sleeve 3 in one direction by subsequent incremental displacements, each corresponding to one tooth of the toothed rim 4a.

Said pair of blocking means may be implemented otherwise, for example the drive sleeve 3 may have different number of the resilient arms, in particular a single arm or more than two arms. Alternatively, as in the embodiment shown in FIG. 5b, a toothed rim—the toothed rim 3b—may be formed on the drive sleeve 3 and the mounting member 4 may have at least one ratchet arm—the ratchet arm 4b.

The drive sleeve 3 has also a flange 3c which, once the drive sleeve 3 has been inserted into the drive mechanism, abuts a ledge 4c formed on the mounting member 4. Consequently, the drive mechanism is stably fastened in the mounting member 4 and upon installation of the drive mechanism in the applicator housing any undesired axial movement of the drive sleeve 3 is prevented. The above described components are indicated in FIG. 3.

The mounting member 4 has also an internal resilient projection 4e allowing the piston rod guide 5 to be inserted into the mounting member 4 from the distal end, along the proximal direction and preventing the piston rod guide 5 from sliding out from the mounting member 4 in the opposite direction. Preferably, the projection 4e is made of a polymer. During assembly of the drive mechanism, a flange 5a formed on the piston rod guide 5 passes through the projection 4e due to its resilience. The projection 4e may have e.g. a triangular cross-section in order to make the insertion of the piston rod guide 5 into the mounting member 4 easier. Once the drive mechanism has been assembled, the piston rod guide 5 abuts the projection 4e, which may extend along the whole circumference of the mounting member 4 so as to form a circumferential shoulder. Alternatively, the projection 4e may only extend along a part of the internal circumference of the mounting member 4.

The housing 7 has an indentation 7a in which a radially outermost part 4g of the mounting member 4 is fitted during the assembly. This arrangement blocks axial movement of the mounting member 4 once installed in the housing 7. Other methods of blocking the axial movement of the mounting member 4 in the housing are also possible. For example, the housing may have projections (ledges) between which the radially outermost part 4g is fitted tightly. In such case, due to the resilience of the material, the mounting member may be pressed through said projection (ledge) during the assembly.

FIG. 3 presents a longitudinal section of a fragment of an applicator in which the described above drive mechanism is mounted. Due to the described design, the assembly of the drive sleeve 3, the piston rod 2, and the piston rod guide 5 is secure within the mounting member 4 and any undesired axial movement is prevented. After securing the assembled drive mechanism in the housing 7, the piston rod 2 is the only element which can move axially and it is displaced distally during medicament delivery, while both the piston rod guide 5 and the drive sleeve 3 are axially immovable.

As can be seen in FIGS. 5a and 5b, the mechanism, once mounted in the housing 7, is prevented from rotating by cooperation of at least one projection 7b, located radially on an internal surface of the housing 7, with at least one indentation 4f located on the circumference of the mounting member 4. In FIG. 4, showing the mechanism mounted in the housing 7 in an isometric view (a part of the housing 7 is removed for the sake of clarity), one indentation 4f engaged with one projection 7b may be seen. FIG. 5b presents the cooperation of the two mutually opposite projections 7b with the respective indentations 4f. As the mounting member 4 cannot rotate in the housing 7, the piston rod guide 5, which is coupled to the housing 7, is also blocked in rotation after the assembly and the piston rod 2 is guided to move axially.

FIG. 5a, presenting a cross-section of the drive mechanism according to the first embodiment, shows the toothed rim 4a cooperating with the ratchet arms 3a. In the presented embodiment, the toothed rim 4a is formed on the mounting member 4 and the two ratchet arms 3a are disposed on the drive sleeve. FIG. 5b shows another embodiment, in which the toothed rim 3b is located on the drive sleeve 3 and the cooperating ratchet arms 4b are disposed on the mounting member 4.

The piston rod guide 5 and the mounting member 4 are provided with cooperating coupling elements preventing the piston rod guide 5 from rotation relative to the mounting member 4 after assembly—see FIG. 6 and FIG. 3. In the embodiment shown in these figures said coupling elements are formed by a flange 5a (shown in FIG. 3) of the piston rod guide 5, two radial projections 5b and two indentations 4d (shown in FIG. 6). The projections 5b are formed on the circumference of the flange 5a and the two indentations 4d are formed in the mounting member 4. The projections 5b and the indentations 4d have mutually corresponding shapes so that they can be coupled during assembly by insertion of the piston rod guide 5 into the mounting member 4 in the proximal direction. After the final assembly of the mechanism, i.e. once the indentations 4d and the projections 5b have been coupled, the piston rod guide 5 cannot rotate relative to the mounting member 4. There may be at least one indentation 4d and any number of the projections 5b, provided that the number of the projections 5b is not larger than the number of the indentations 4d.

Therefore, when the drive mechanism is assembled in the applicator the piston rod guide 5 is immovable relative to the housing 7. The piston rod guide 5 has a central, non-circular aperture 6 with a shape matching the non-circular cross-section of the piston rod 2. This aperture can be seen for example in FIG. 7 presenting the components of the drive mechanism in an isometric view.

The piston rod 2, when coupled with the piston rod guide 5 through its aperture 6, can only move axially and cannot rotate as the piston rod guide 5 does not rotate. The shape matching of the piston rod 2 and the aperture 6 may be implemented for example as flattenings on the surface of the piston rod 2 engaging a planar internal surface of the aperture 6, indentations on the surface of the piston rod 2 engaging projections on the piston rod guide aperture 6 or other formations engaging a suitable shape of the aperture 6, preventing rotation of the piston rod 2 within the aperture 6. In FIG. 7 an exemplary projection 5d engaging the internal surface of the aperture 6 is shown. In FIG. 7 also ribs 5c are indicated (also to be seen in FIG. 1) enhancing the stiffness and stability of the piston rod guide 5, which may be made of a polymer by injection moulding.

The piston rod 2 has a thread 2b and projections for fixing the piston rod foot 2a. This connection may also be realised otherwise, by methods known in the art. The piston rod 2 may also be made integrally with the piston rod foot 2a.

FIGS. 8-9 show still another embodiment, where the piston rod guide 5 is coupled to the mounting member 4 by means of numerous projections 5b′ cooperating with indentations 4d′ and alternating projections 4e′ of the mounting member 4. The numerous projections enable a more precise positioning of the piston rod guide 5 with respect to the mounting member 4 and, as a result, a more precise adjusting of the initial axial position of the piston rod 2. The width of the projections 4e′ is selected so as to match the distance between the subsequent projections 5b′ of the piston rod guide. FIG. 8 shows a cross-section of this embodiment and FIG. 9 shows the components of the embodiment of the drive mechanism of FIG. 8 in an isometric view.

Subsequent steps of the method of assembling the drive mechanism according to the invention are presented in FIGS. 10a-10c.

The assembly of the drive mechanism according to the invention starts with sliding the piston rod guide 5 onto the piston rod 2 having the piston rod foot 2a fixed on its distal end. The piston rod guide 5 is being slid from the proximal side. This step is shown in FIG. 10a. Alternatively, the piston rod guide 5 can be slid onto the piston rod 2 before the piston rod foot 2a is fixed at its distal end. In this case the piston rod guide 5 may be slid from any side. Further (FIG. 10b), the drive sleeve 3 is being screwed onto the piston rod 2. The drive sleeve 3, with the piston rod 2 having the piston rod guide slid onto it, is then being inserted into the mounting member 4. Before this step (shown in FIG. 10c), the angular position of the piston rod guide 5 is being set. To that purpose, the piston rod guide 5 is being rotated which enables the setting of the initial axial position of the piston rod 2. The rotation of the piston rod 2 along with the piston rod guide 5 causes the axial displacement of the piston rod 2 due to its threaded engagement with the drive sleeve 3. When the assembly of the drive sleeve 3, the piston rod 2 and the piston rod guide 5 is engaged with the mounting member 4, the drive sleeve 3 is coupled with the mounting member 4 by the pair of the blocking means 3a and 4a (or alternatively 3b and 4b). In this step the coupling elements (5a, 5b, 5d) of the piston rod guide 5 and the mounting member 4 are also coupled fixing the position of the piston rod guide 5 in the mounting member 4 as well as the initial axial position of the piston rod 2. This axial position should be adjusted to the initial position of the piston in the cartridge with medicament to be delivered by means of the applicator. As the initial position of the piston rod foot 2a can be adjusted, the drive mechanism may be used with many different kinds of the medicament cartridges having the pistons located initially in the different axial positions. An important feature of the method according to the invention is that the drive sleeve 3 together with the piston rod guide 5 and the piston rod 2 are inserted into the mounting member 4 from the side of the distal end. Preferably, the drive sleeve 3 with the piston rod 2 and the piston rod guide 5 are inserted into the mounting member 4 by overcoming the resistance of the internal resilient projection 4e of the mounting member 4. Alternatively, in each step the assembly may be realised by movement of the other of the connected components in a direction opposite to the direction indicated in the respective figures. In FIG. 10c for example the drive sleeve 3, the piston rod 2 and the piston rod guide 5 are inserted into the mounting member 4 from the distal side. However, it is also possible to realise this step the way round, i.e. the drive sleeve 3, the piston rod 2 and the piston rod guide 5 may be motionless and the mounting member 4 may be mounted thereon in the direction opposite to the direction marked in FIG. 10.

After the above steps of the assembly have been completed, the drive mechanism is being located within the applicator housing, where it is being blocked axially by securing the mounting member 4 in the indentation 7a.

1. A drive mechanism for an applicator for delivering multiple doses of medicament, the drive mechanism having a distal end facing the needle end of the applicator, the drive mechanism comprising:
an axially moveable piston rod (2) having a non-circular cross-section and provided with an external thread (2b),
a mounting member (4) provided with one member of a pair of blocking means, said pair comprising a toothed rim (4a, 3b) and at least one ratchet arm (3a, 4b);
a drive sleeve (3) having an internal thread coupled with the external thread of the piston rod (2), the drive sleeve (3) being provided on the distal end with the other member of said pair of blocking means,
wherein the blocking means of the mounting member (4a, 4b) and the blocking means of the drive sleeve (3a, 3b) cooperate radially during rotation of the drive sleeve (3), the rotation causing translation of the piston rod (2),
characterized in that
the drive mechanism further comprises a piston rod guide (5) provided with a central aperture (6), in which the piston rod (2) is axially movable and blocked in rotation,
the piston rod guide (5) and the mounting member (4) being provided with coupling elements (5a, 5b, 4d) for preventing rotation of the piston rod guide (5) relative to the mounting member (4).

2. The drive mechanism according to the claim 1, characterized in that the mounting member (4) is provided with the member of said pair of blocking means constituted by the internal toothed rim (4a) and the drive sleeve (3) is provided with the member of said pair of blocking means constituted by the at least one ratchet arm (3a).

3. The drive mechanism according to the claim 1, characterized in that the mounting member (4) is provided with the member of said pair of blocking means constituted by the at least one ratchet arm (4b) and the drive sleeve (3) is provided with the member of said pair of blocking means constituted by the external toothed rim (3b).

4. The drive mechanism according to any of the preceding claims, characterized in that the coupling elements are constituted by a flange (5a) of the piston rod guide (5), the flange (5a) being provided at its circumference with at least one projection (5b, 5b′) and by at least one indentation (4d) of the mounting member (4).

5. The drive mechanism according to any of the preceding claims, characterized in that the mounting member (4) comprises an internal resilient projection (4e) allowing the piston rod guide (5) to be inserted into the mounting member (4) from the distal end and preventing the piston rod guide (5) from sliding back out of the mounting member (4).

6. The drive mechanism according to claim 5, characterized in that, the internal projection (4e) is a circumferential shoulder.

7. A method of assembling the drive mechanism of any of the preceding claims, the method comprising the following steps:
a step of sliding the piston rod guide (5) onto the piston rod (2);
a step of screwing the drive sleeve (3) onto the piston rod (2);
a step of inserting the drive sleeve (3) together with the piston rod (2) and the piston rod guide (5) into the mounting member (4),
characterized in that the step of the inserting the drive sleeve (3) together with the piston rod (2) and the piston rod guide (5) into the mounting member (4) is realized from the side of the distal end, and in that the drive sleeve (3) is coupled with the mounting member (4) by the pair of blocking means comprising a toothed rim (4a, 3b) and at least one ratchet arm (3a, 4b) while the coupling elements (5a, 5b, 4d) of the piston rod guide (5) are simultaneously engaged with the mounting member (4),
wherein
an axial position of the piston rod (2) in relation to the drive sleeve (3) is pre-set by rotation of the piston rod guide (5) relative to the mounting member (4) prior to the coupling of the blocking means (3a; 4a; 3b; 4b) and engaging the coupling elements (5a, 5b, 4d).

8. The method according to the claim 7, characterized in that the step of inserting the drive sleeve (3) with the piston rod (2) and the piston rod guide (5) into the mounting member (4) involves overcoming resistance of the internal resilient projection (4e) of the mounting member (4).
